(19) United States
(12) Patent Application Publication
(10) Pub. No.: US 2023/0240593 A1
(43) Pub. Date: Aug. 3, 2023

(54) METHOD FOR THE OPTICAL EXAMINATION OF HUMAN NAILS AND SYSTEM FOR IMPLEMENTATION OF THE METHOD

(71) Applicant: MEDINER GYÓGYSZERKERESKEDELMI, MARKETING ÉS SZOLGÁLTATÓ KFT., Debrecen (HU)

(72) Inventors: Rolland GYULAI, Pécs (HU); Júlia SZEBÉNYI, Kaposvár (HU); Tamás SZÉPE, Szeged (HU); András BÁNHALMI, Szeged (HU); Vilmos BILICKI, Szeged (HU); Péter GASZNER, Debrecen (HU); Róbert DÉVÉNYI, Budapest (HU); Zsolt István ÁCS, Debrecen (HU); Péter LÕDÁR, Mezõtúr (HU)

(73) Assignee: MEDINER GYÓGYSZERKERESKEDELMI, MARKETING ÉS SZOLGÁLTATÓ KFT., Debrecen (HU)

(21) Appl. No.: 17/918,929

(22) PCT Filed: Apr. 19, 2021
(86) PCT No.: PCT/HU2021/050024

§ 371 Date: Oct. 14, 2022

(30) Foreign Application Priority Data
Apr. 22, 2020 (HU) P2000138

(51) Int. Cl. A61B 5/00

(52) U.S. Cl. CPC A61B 5/449; A61B 5/0077; A61B 5/6826; A61B 5/6838

(57) ABSTRACT

System for the optical examination of human nails that has a camera and a power source or a smartphone provided with a connection to these, a converter connected to the smartphone, which converter contains a housing and a measuring probe, the housing contains a microcontroller, the measuring probe contains at least one macro lens and at least one polarising filter, at least two light sources and a polarising filter film in front of some of the light sources arranged in such a way that the axes of polarisation of the polarising filter films and of the polarising filter are rotated by 90 degrees as compared to each other, where due to the various light sources the system is adapted to use various illumination modes and the smartphone is connected to a neural network. Also, a method for the examination of nails for diagnostic purposes with such devices.

### THE FIELD OF THE INVENTION

The object of the present invention relates to a method for the optical examination of human nails and to a system for the implementation of the method.

### THE STATE OF THE ART

The human nail (unguis) is a slightly convex, keratinous plate located on the rear side of the final, distal phalanx of the fingers and toes, which is secured to a modified part of the dermis of the skin, which is called the nail bed.

The nail plate protrudes freely from the nail bed, its rear and side edges are embedded in a frame formed of groove-shaped folds of skin. The skin edges protruding above this frame are called the cuticle. The thickest part is the nail body, this becomes sharper towards the edges, and its softer, rear end penetrates deep into the cuticle, where it forms the nail root. The material of the nail is produced by the matrix cells located at the root of the nail. The external surface of the nail body is smooth, on the underside there are longitudinal grooves that correspond to the few-millimetres high grooves or matrix crests (cristae matricis unguis) in the dermis. These crests consist of compacted dermal tissue, mixed with a multitude of flexible fibres, these contain the blood vessels and nerves that create the pink tone of the nail, and due to their large number injuries to the nail bed are extremely painful.

Characteristic pathological changes to the nail include injuries caused by physical impact and infections caused by microorganisms (e.g. fungi, bacteria). Pathological changes to the nail may also originate from conditions not originating in the nail (e.g. cardiovascular, liver and kidney diseases). Such changes may include, for example:
The lunula (lighter, crescent-shaped part at the base of the nail) may become reddish. Sharply defined erythema appearing in the region of the lunula on all nails, usually with dominance on the thumb has been identified in connection with the following conditions: high blood pressure, angina pectoris, atherosclerosis, heart failure, myocardial infarction (1, 3).
Clubbing (watch-glass nails). Due to proliferation of the soft tissue under the nail plate the shape of the nail develops to resemble a watch glass. The nail plate becomes convex in shape and grows larger. In most cases the background of these symptoms includes lung disease (chronic bronchitis—COPD, bronchiectasia, carcinoma, fibrosis), however this condition may also be idiopathic or hereditary (1).
Capillary bleeding. Capillary bleeding located at the proximal part of the nail plate (splinter haemorrhage) suggests inflammation of the endocardium (endocarditis). This capillary bleeding appears in the form of small, thick 1-2 mm wide macules. (5)
Apparent leukonychia (“half and half nails”). The appearance of the nails is split up into two parts with a well-defined transverse border. The proximal portion appears white and the distal portion appears pink-red-brown. This occurs in the case of kidney diseases (e.g. uremia) (1).
Apparent leukonychia (“Terry's nails”). In the case of this abnormality the nails appear white with a “ground glass” appearance. The end of the change of colour is located 1-2 mm proximally from the hyponychium, with a pink-brown band following the white band. The lunula characteristically disappears, and in numerous cases teleangiectasia may be observed on the area of skin around the proximal nail. This characteristically suggests a background of liver disease (e.g. fibrosis, cirrhosis) (1, 2).
Onycholysis (separation of the nail plate from the nail bed). The shape and extent of the onycholysis provides assistance in the diagnosis of the nail symptoms.

Onycholysis with an irregular, corrugated edge may be observed in the case of nail fungus. Distal onycholysis occurs in the case of dermatophyte infection and proximal onycholysis occurs in the case of candida infection. Onycholysis with a regular edge may be usually observed in the case of nail trauma. (4)

### REFERENCES

1. Rubin A. I., Holzberg M., Baran R.; Physical Signs, Baran & Dawber's Diseases of the Nails and their Management, Fifth Edition; 7 Dec. 2018

2. Terry R.; White nails in hepatic cirrhosis. Lancet. 1954; 266:757-759.

3. Terry R.; Red half-moons in cardiac failure. Lancet. 1954; 267:842-844.

4. Piraccini B. M., Balestri, R., Starace M., Rech G.; Nail digital dermoscopy (onychoscopy) in the diagnosis of onychomycosis. J Eur Acad Dermatol Venereol. 2013; 27(4):509-513.

5. Chong Y., Han S. J., Rhee Y. J., Kang S. K., Yu J. H., Na M. H.; Classic peripheral signs of subacute bacterial endocarditis. Korean J Thorac Cardiovasc Surg.

It should be noted that numerous other abnormalities in addition to the examples above may also occur in the nails that originate from non-nail diseases.

From the abnormalities mentioned above it is obvious that the examination of the nails is an important tool in determining the state of health of patients. A specialist is required in order to examine the nails with the naked eye and due to the exceptionally varied forms of the abnormalities even specialists can make mistakes. In addition to this some of these abnormalities cannot be seen by the naked eye, or it may be difficult to differentiate between similar abnormalities when examining the nails with the naked eye.

Document publication number U.S. Pat. No. 6,631,199 B1 presents a system that scans the nail bed using polarised light and a reference light and then maps out the shape of the nail on the basis of the reflected light and stores it in a memory. Using this the photograph made of people's nails will be suitable later on for determining the given person's identity, similarly to fingerprints.

Patent document with publication number WO2005/081880 A2 presents a method with which certain elements in the blood may be examined through the nails using Raman spectroscopy, in this way a patient's blood sugar level may be determined in a non-invasive way. A disadvantage of the method is that Raman spectroscopy is a complex method in which it may also be necessary to control the temperature. Furthermore, the method it is only capable of analysing certain components contained in the blood.

Similarly, patent document number EP2922469 B1 describes a device suitable for testing blood. The document discloses a device that tests the blood through the skin using an oscillating electric field, and measures the amount of glucose present in the patient's blood.

The dermatoscope is a commonly used medical device for examining abnormalities of the skin. The following publication presents the use of the dermatoscope for the examination of nails: Pellacani, G. et al., JEADV 2019, 33, 2355-2361, DOI: 10.1111/jdv.15790. The patients' fingernails or toenails are examined with a dermatoscope using polarised white light and with a dermatoscope using polarised white light combined with polarised red light, where the polarised red light is produced using an LED located in the base of the device, onto which the patient's finger or toe is placed. The study showed that a dermatoscope that also uses red light facilitates the identification of the main abnormalities of the nails and of the vascular abnormalities related to these.

The disadvantage of the device is that a specialist is required in order to recognise the visible abnormalities, additionally it is only able to identify diseases of the nails. Furthermore, it only reveals the precise details of the abnormalities and the related vascular changes that can generally be seen with the naked eye. Another disadvantage of the device is that in order for certain parts of the nail to be more visible it may be necessary to move the fingers or toes and photograph them from various angles. This means that during the use of the device, as a result of the movement of the fingers or toes, or simply because the patient's finger or toe does not completely cover the light source located on the base of the device, the light may shine out of there into the patient's or operator's eyes and cause discomfort or even injury to the eye.

Due to the above there is a need for a system that can be used to perform a diagnosis through the optical examination of the nails even without the involvement of a specialist. It is also necessary for the abnormalities to be identified at such early stages when there are no signs of them that may be seen with the naked eye. Additionally it is also necessary to be able to identify certain health conditions in patients that are not directly related to the nails via the examination of the nails and the area around the nails.

### BRIEF DESCRIPTION OF THE INVENTION

The present invention is based on the recognition that using light sources emitting light at various wavelengths in various illumination modes the nail and the areas around the nail may be sufficiently mapped in order to be able to identify nail diseases and certain other underlying conditions with the use of a neural network, even in their early stages when the abnormalities caused by them are still almost invisible. Furthermore, by securing the finger or the toe with a securing device the examined area may be mapped in its entirety without moving either the nail or the device, and thereby it is possible to prevent the light from the light source getting into the eyes of the patient or operation personnel. In addition, when a finger/toe securing device is used the device acts as a shield against external sources of light, so these do not disturb the photographing process.

In accordance with the above the present invention relates to a system that has a camera and a power source or a smartphone provided with a connection to these, a converter connected to the smartphone, which converter contains a housing and a measuring probe connected to it, the housing contains a microcontroller, the measuring probe contains at least one macro lens and at least one polarising filter, at least two light sources and a polarising filter film in front of some of the light sources arranged in such a way that the axes of polarisation of the polarising filter films and of the polarising filter are rotated by 90 degrees as compared to each other, characteristics of which is that as a result of the various light sources the system is adapted to use various illumination modes and the smartphone is connected to a neural network.

A preferred embodiment of the system according to the invention contains a finger clamp connected to the measuring probe adapted for securing a finger or a toe.

A more preferable embodiment of the system according to the invention contains a transillumination attachment with an additional light source, which transillumination attachment is connected to the microcontroller in the housing and connected to the finger clamp on the side opposite the nail of the finger to be clamped.

According to another preferable embodiment of the system according to the invention the measuring probe contains at least two macro lenses.

According to another preferable embodiment of the system according to the invention the light sources are multichannel light sources, preferably at least three-channel light sources, more preferably at least four-channel light sources, most preferably at least six-channel light sources.

According to a more preferable embodiment of the system according to the invention the colours of the multichannel light sources are selected from among the following: red, green, blue, white, yellow, UV.

According to another preferable embodiment of the system according to the invention at least six, preferably eight light sources are located in the measuring probe.

According to another preferable embodiment of the system according to the invention polarising filter film is placed in front of a half of the light sources located in the measuring probe, or in the case of an odd number of light sources one more or one less of the light sources has a polarising filter film than the number of light sources without a polarising filter film.

Furthermore the present invention relates to a method for the optical examination of fingernails using the system according to the above, which contains the following steps:
a) connecting the smartphone to the converter or using the converter integrated with the smartphone;
b) placing the patient's finger or toe in an immobile position in front of the system's measuring probe in a position suitable for taking photographs;
c) adjusting the focus directed at the nail of the finger or toe of the patient to be examined on the smartphone;
d) illuminating the fingernail or toenail to be examined using at least two different illumination modes and the taking of photographs of the fingernail or toenail while the various illumination modes are being applied;
e) sending the photographs to the neural network;
f) the neural network determining the patient's health condition on the basis of the database it has at its disposal on the basis of the photographs that have been taken.

In the case of a preferred embodiment of the method according to the present invention in step b) the patient's finger or toe is secured in an immobile position using a finger clamp.

In the case of a preferred embodiment of the method according to the present invention one of the at least two different illumination modes used in step d) is the transillumination mode.

In the case of a preferred embodiment of the method according to the present invention during step d) photographs are also taken in an illumination mode with large focus.

In the case of a preferred embodiment of the method according to the present invention in step d) a multichannel light source (23) is used in the case of the individual illumination modes.

### IN THE FIGURES

FIG. 1a shows a perspective top view of the converter according to the invention;

FIG. 1b shows a perspective bottom view of the converter according to the invention;

FIG. 2 shows an exploded view of the measuring probe according to the invention;

FIG. 3 shows the finger clamp according to the invention with the transillumination attachment;

FIG. 4 shows the absorption capability of certain constituent elements of the human body.

### DETAILED DESCRIPTION OF THE INVENTION

The essence of the system according to the invention is that by using light sources emitting light at various wavelengths in various illumination modes, by securing the fingernail or toenail, and with the use of a neural network the diseases of the examined fingernail or toenail and certain underlying conditions may be determined.

In the context of the present specification nail also means toenail, unless something else is expressly written in the given section of text.

In the context of the present invention underlying condition is understood to mean diseases that cause abnormalities in various parts of the body, therefore, even on the nails and the areas around the nails, but which are not nail-specific diseases. For example, a high blood sugar level or high blood pressure can cause changes in the entire vascular system, and differences that can be seen on the nails, in addition the nail may become deformed due to certain heart and liver diseases. Also, certain deficiency conditions and hormone fluctuations cause changes to the nails.

In the context of the present invention, a multichannel light source is understood to mean a light source that is capable of emitting light at various wavelengths, even simultaneously.

In the context of the present invention the colour red is understood to mean light of a wavelength that has its emission spectrum maximum between 615-635 nm.

In the context of the present invention the colour green is understood to mean light of a wavelength that has its emission spectrum maximum between 515-535 nm.

In the context of the present invention the colour blue is understood to mean light of a wavelength that has its emission spectrum maximum between 455-475 nm.

In the context of the present invention the colour white is understood to mean light of a wavelength that has a colour temperature of between 5800-8200 K.

In the context of the present invention the colour yellow is understood to mean light of a wavelength that has its emission spectrum maximum between 585-605 nm.

In the context of the present invention the colour UV is understood to mean light of a wavelength that has its emission spectrum maximum between 390-410 nm.

In the context of the present invention a smartphone is understood to mean a device that has at least the following elements:
display, touchscreen or keyboard for control, processor and memory for running the application, power supply, furthermore it should be adapted for wired or wireless communication and data transfer.

In the context of the present invention power supply is understood to mean a device capable of supplying electricity. Such include batteries, rechargeable batteries, and an electricity network.

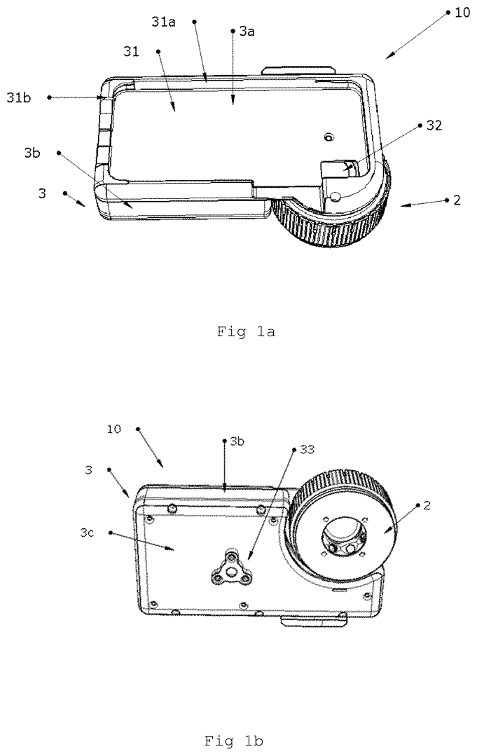

FIG. 1a also depicts one of the main elements of the system according to the invention, the converter, which in its entirety is marked with reference sign 10, the main parts of which converter 10 are the measuring probe 2 and the housing 3. The system also contains a smartphone and a neural network (not depicted).

A socket 31 in which the smartphone may be secured is formed in the upper part 3a of the housing 3. A gap 32 ensures that the nail is visible to the camera located in the smartphone. The upper part 3a forms a platform for securing the microcontroller electronics (not depicted), and for securing the measuring probe 2 under the gap 32. An intermediate part 3b and a lower part 3c (depicted in FIG. 1b) encompass the electronic components, the cables, and provide a space for the electronics for a transillumination attachment 5 (not depicted). A support frame securing point 33 (depicted in FIG. 1b) may be located on the lower part 3c. The elements of the housing 3 may each be separately attached to one another, with screws, for example, or the elements may be formed in such a way so that they can be clicked into one another, or their connection to one another may be solved in other ways known to the person skilled in the art. The material of the housing 3 may be, for example, metal used for medical purposes, glass or plastic.

The socket 31 is formed in such a way that the smartphone can be inserted under a flange 31a and then the inserted smartphone may be secured with an end closing element 3b. Naturally, the use of a different device provided with a camera is not excluded during the use of the system, in the case of which the camera's objective system may be positioned above the gap 32. If the camera used does not have an integrated display (such as in the case of a smartphone), a display must be built into the converter 10 or a display must be connected to the camera via a wired or wireless connection. The converter 10 may be integrated with a camera and if it is not connected to a device with an energy store, an energy store may be built into the converter 10, or it may be connected to an external energy store or power network in a way obvious to the person skilled in the art.

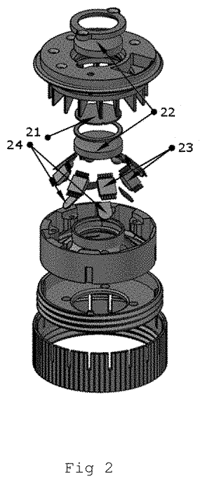

The measuring probe 2 shown in FIG. 2 secures the optical elements required for the operation of the device 10. A polarising filter 21 is positioned immediately under the gap 32, and under it at least one macro lens with light sources 23 under this; polarising filter film 24 is placed in front of at least some of the light sources 23 in such a way that the axis of polarisation of the light is rotated by 90 degrees between the polarising filter 21 and the polarising filter films 24. The light sources 23 are positioned in such a way that if the macro lens 22 permits it they face downwards and illuminate the space from as many sides as possible, thereby avoiding certain protrusions of the nail causing bothersome shadows. For example, the light sources 23 are at an angle of 45° to the optical axis, otherwise known as the axis of symmetry of the macro lens 22. The threaded inner part 2a of the measuring probe 2 is provided with a threaded external part 2b so that in this way the distance between the finger or toe being examined and the camera can be adjusted. The external material of the measuring probe 2 may be metal used for medical purposes, glass or biocompatible plastic (e.g. PTFE—polytetrafluoroethylene, PEEK—polyether ether ketone, PET—polyethylene terephthalate, PC—polycarbonate, PLA—polylactic acid, etc.). In the case of the measuring probe 2 it is important to be able to sterilise it, as the element may come into contact with the patient.

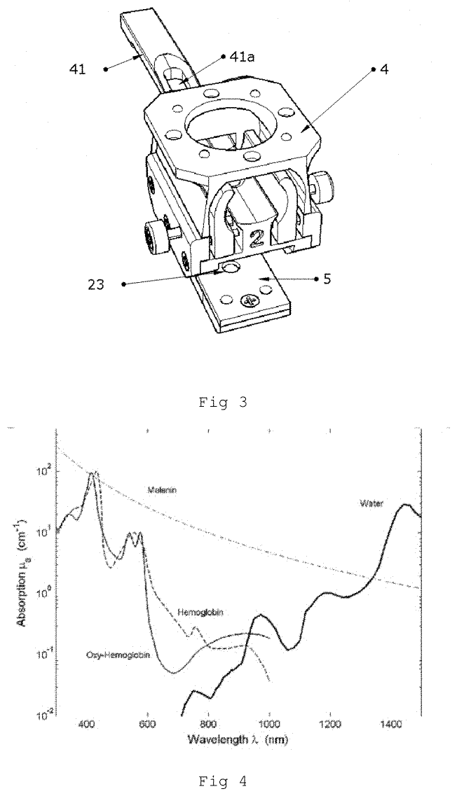

Preferably a finger clamp 4 illustrated in FIG. 3 is connected to the converter 10 for the efficient operation of the device.

The transillumination attachment 5 may be connected to this finger clamp 4. Essentially the finger clamp 4 serves for securing the finger or toe under the gap 32 and keeping it there. The securing of the finger may take place using two lateral plates (not depicted) as in the case of a vice. A lifting wedge 41 may be inserted under the fingertip, which is provided with a slot 41a for the light emitted by the transillumination attachment 5. The finger clamp 4 and the transillumination attachment 5 may be secured to each other with magnets and the finger clamp 4 may also be secured to the measuring probe 2 with magnets. Naturally the elements may also be connected to each other with screws or flanges that fit into each other. The material of the finger clamp 4 may be metal used for medical purposes, glass or biocompatible plastic. In the case if the finger clamp 4 it is important to provide the possibility of disinfection, as this element comes into contact with the patient. Naturally, as is known to the person skilled in the art, the finger clamp 4 may be formed in innumerable sizes depending on whether fingers or toes are to be secured, or whether the patients are adults or children.

The transillumination attachment 5 contains at least one light source 23, with which the examined finger or toe may be illuminated from below. In addition to the positioning of the finger or toe, the purpose of the finger clamp 4 and the associated lifting wedge 41 is to prevent the light of the light sources 23 getting into the eyes of the patient or the operating personnel during the use of the converter 10. The material of the transillumination attachment 5 may be metal used for medical purposes, glass or plastic.

During the use of the system the patient's finger or toe is placed in the finger clamp 4 or onto a flat surface and then the fingernail or toenail is illuminated in various ways in order to map out the physical condition of the fingernail or toenail and the surrounding areas.

Various ways of illumination is understood to mean, for example, illumination with polarised light or with non-polarised light, transillumination and the illumination of the nail bed with a large focus.

In the case of illumination with polarised light a photograph may be taken of the surface of the nail through a polarising filter 21 arranged in front of the macro lens 22. In the interest of obtaining a sufficient degree of definition it must be possible to place the camera of the smartphone sufficiently close up to the surface of the nail to be examined, and it is for this reason that there is a need for the macro lens 22 in addition to the smartphone's own objective.

The size of the visible area preferably approaches 2 cm×2 cm so that the entire surface of a larger nail fits into it. For this the distance of the macro lens 22, the polarising filter 21, the camera and the nail are maintained at fixed values. The illumination takes place with six-channel light sources 23 illuminating through a polarising filter film 24 rotated by 90 degrees as compared to the polarising filter 21 placed in front of the lens, in this way the colours that may be selected are: red, green, blue, white, yellow, UV. The desired effect is achieved if the axes of polarisation of the polarising filter films 24 placed in front of the light source 23 and of the polarising filter 21 placed in front of the camera are rotated by 90 degrees as compared to each other. Preferably four six-channel light sources 23 are positioned around the nail in the interest of even illumination, in this way the photograph taken of the nail will be free of shadows.

The polarised light penetrates in between the layers of the nail and illuminates through the deeper layers instead of the surface.

In the case of illumination performed with non-polarised light the nail surface to be examined is illuminated with the light of non-polarised light sources 23. In the case of this illumination mode, the wavelengths of the light emitted by the light sources 23 corresponds to the following colours: red, green, blue, white, yellow, UV. The intensity of the illumination and its scheduling may be controlled indirectly through the smartphone. It is possible to switch the six-channel light sources 23 off and on using the microcontroller connected by cable to the telephone.

Preferably four six-channel light sources 23 are positioned around the nail in the interest of even illumination, in this way the photographs taken of the nail will be free of shadows.

The non-polarised light illuminates the surface of the nail, so a photograph of that is provided.

In transillumination mode the transillumination attachment 5 is connected to the finger clamp 4. By using the transillumination attachment 5 the nail receives such intense illumination through the lower part of the associated phalanx that makes it possible to take a photograph through the upper surface of the nail even without the use of other light sources 23. By using a four-channel light source 23 for the transillumination the number of the types of examination that may be performed may be significantly increased. The wavelength of the light emitted by the four-channel light source 23 preferably corresponds to the following colours: white, red, green, and blue. The examination may also be performed with any combination of these colours. In this mode the electronics responsible for performing the illumination control all four channels individually, the software running on the smartphone controls the light intensity.

Naturally, the possibility of individually controlling the channels is not excluded in the other forms of illumination either. But, fundamentally, there is no need for this because it is more preferable to use each of the light sources 23 belonging to the illumination mode with the same wavelength of illumination at the same time and then to use another wavelength following this. In addition, in the case of illumination from above, it is sufficient to adjust the light intensity once and then use this setting from that point onwards. Contrary to this, in the case of illumination from below the thickness of the examined finger/toe and of the nail on it changes from finger to finger and from toe to toe, therefore it is also necessary to vary the intensity of the light required for the appropriate transillumination of the finger/toe.

By illuminating the nail from below in transillumination mode it is possible to illuminate the vascular system under the nail.

In the case of a large focus illumination mode magnification is made use of by focussing the field of view onto the nail bed.

The system produces high-resolution photographs through the at least two macro lenses 22 located in the converter each preferably with a magnifying power of 15×, in this way the degree of supplementary optical magnification is 30×. The light sources 23 used in the previous, upper illumination modes may be used for illumination here also. It is preferable to use the light sources 23 used during illumination with polarised light and so, due to the use of polarised light, reflections from shiny skin will not disturb the photograph.

In the case of large focus illumination it is necessary to place another macro lens 22 under the other macro lens 22, with which the capillary vessels under the nail bed may be shown, which has great significance from a medical point of view.

The various illumination modes effectively illuminate the various structural elements of the nail and of the skin around the nail, therefore it is preferable to use as many different illumination modes as possible with the use of light of various wavelengths. It is important, for example, for the polarised light to penetrate into the deeper layers of the nail, so that it is not only the surface abnormalities that are identified. In addition non-polarised light provides a detailed photograph of the surface of the nail. In the case of transillumination, the network of blood vessels under the nail can be revealed in detail, so by using this illumination mode abnormalities of the area under the nail can be displayed. In the case of large focus illumination the nail bed and the small capillary blood vessels located there may be examined in detail, which has great significance from a medical point of view.

The use of light of various wavelengths is justified because the different tissue elements, such as haemoglobin, protein, keratin and water all have different light absorption capabilities, therefore, in the case of illumination with light of various wavelengths, different elements will be emphasised in the individual photographs. FIG. 5 shows the absorption characteristics of a number of such tissue elements.

Naturally, the use of other illumination methods is not excluded, for example, magnification of other areas of the nail apart from the nail bed, magnification of the other edges, possibly the use of an even greater degree of magnification. In addition the use of light sources 23 emitting light at other wavelengths is not excluded either in the context of the present invention.

A neural network assesses the photographs taken in the individual illumination modes. The neural network is trained on the basis of a database that is set up in advance. The database contains the basic data of the examined patients, such as skin colour, age, sex, etc., and their health data, such as blood pressure, blood sugar level, allergies, underlying illnesses, etc. and it links these data with the photographs taken of the fingernails and toenails of the individuals in each of the illumination modes. Using this database the neural network is able to identify deeper relationships, even those that human knowledge was not aware of to date, between the individual conditions and their effects on the fingernails, toenails and the surrounding areas.

Using this knowledge it is able to identify conditions even in their early stages. Naturally, with the continuous expansion of the database, the neural network will be able to identify an increasing number of diseases and its efficiency will also increase. The smartphone sends the photographs taken during the examination via a wired or wireless network to the server on which the neural network is running.

Concrete training of the neural network with a training database took place as follows:

Illuminating with one or several different colours one or more photographs were made of the patient's nail with the device. These photographs were annotated by specialists. Annotation is understood to mean the marking of certain areas on the photographs and assigning textual information to the markings.

In practice this meant, for example, that the specialist marked out a rectangle with its corner points on photographs of the nail illuminated with the colours red, white, etc. and designated it by stating what diagnostically relevant features or classes can be seen in the affected area, such as bruising or nail fungus. In addition to this it was also possible to give information valid for the entire photograph, such as the patient whose nail is shown in the photograph has high blood pressure, or other parameter to be recognised.

The use of neural networks is known of to the person skilled in the art. Handbooks and studies presenting neural networks include the following:
Márta Altrichter, Gábor Horváth, Béla Pataki, György Strausz, Gábor Takács, József Valyon, Neural Networks, Hungarian Edition Panem KönyvkiadóKft., Budapest, 2006;
del Aguila M. R., Requena I., Bernier J. L., Ros E., Mota S. (2004) Neural Networks and Statistics: A Review of the Literature, Soft Methodology and Random Information Systems. Advances in Soft Computing, vol 26. pp 597-604 Springer, Berlin, Heidelberg;
Asifullah Khan, Anabia Sohail, Umme Zahoora, and Aqsa Saeed Qureshi (2019) A Survey of the Recent Architectures of Deep Convolutional Neural Networks, Artificial Intelligence Review (Springer Nature), January, 2019.

During the examination the measuring method must contain the following steps:
Connecting a smartphone to the converter 10;
Securing the fingernail or toenail in an immobile position;
Adjusting the focus on the smartphone or on the built-in display;
Illuminating the fingernail, toenail using the individual illumination modes and the taking of photographs of the fingernails and toenails in the course of the various modes of illumination;
The sending of the photographs to the neural network;
The Neural network determines the patient's health status using the photographs that have been taken on the basis of the database at its disposal.

During the method the smartphone is placed on the converter 10 so that photographs can be taken of the patient's fingernails, toenails using the smartphone's camera. Following this the camera focus is adjusted to the fingernail, toenail positioned in advance so that clear photographs can be taken.

Following this the converter 10 takes photographs of the fingernail, toenail according to each of the illumination modes that can be used by the measuring probe 2. Then the smartphone sends the photographs that have been taken to the neural network, which surveys the patient's status of health using the database that was set up previously and the previous training procedures on the basis of the fingernail and the toenail.

It is preferable to use the finger clamp 4 when securing the patient's fingernail, toenail, in this way light is not dispersed during the examination, and the external light does not disturb the examination. In addition there is less chance of the fingernail or toenail moving during the examination. In the case of the use of the finger clamp 4, the transillumination attachment 5 may also be connected, so, in this way, photographs may also be taken in transillumination mode.

If the measuring probe 2 contains at least two macro lenses 22 photographs may also be taken in large focus illumination mode.

It is preferable to use multichannel light sources 23 and take separate photographs with the various wavelengths of light emitted by the multichannel light sources 23 in the case of each illumination mode.

The use of the system is not restricted to human patients, the nails or claws of animals may also be examined using the system according to the invention.

### EXAMPLES

### Example 1

### System for the Examination of Nails

The system serving for examining nails consists of a neural network, a smartphone and the converter 10 depicted in FIG. 1.

The converter 10 consists of a measuring probe 2 and a housing 3, which has a socket 31 for connecting a smartphone to the converter 10 via a USB cable. Through this the battery of the smartphone supplies the converter 10 with energy.

The measuring probe 2 contains a polarising filter 21, two macro lenses under it and four six-channel (red, green, blue, white, yellow and UV) LED light sources 23 provided with a polarising filter film 24 for illumination with polarised light. Furthermore, it contains four six-channel (red, green, blue, white, yellow and UV) LED light sources 23 for illumination with a non-polarised light source 23. Using the two macro lenses 22 the converter 10 is able to take photographs in large focus illumination mode. In order to achieve polarisation the polarising filter films 24 and the polarising filter 21 are rotated by 90 degrees as compared to each other.

Using an application running on the smartphone the patient's relevant data are provided, such as sex, age, occupational harms, harmful habits (smoking, alcohol consumption), health status, regularly taken medication and other data important from the point of view of the examination. Following this the particular finger or toe of the patient that is to be examined is set in the application and then that finger or toe is secured on the desktop. The valid data protection regulations are taken into consideration when the patient's data are recorded.

After placing the device above the patient's finger or toe the focus is adjusted and by starting the application photographs are taken in all of the illumination modes. By running the photographs that have been taken through the neural network, the neural network uses the database to search for relationships between the patterns visible in the photographs, physical abnormalities and the disease-photograph pairs contained in the databases.

### Example 2

### System for the Examination of Nails

The system according to example 1, where the measuring probe 2 contains white LED light sources 23.

### Example 3

### System for the Examination of Nails

The system according to example 1, where the measuring probe 2 contains two-channel (red, green) LED light sources 23.

### Example 4

### System for the Examination of Nails with Finger Clamp 4

The system according to example 1 is supplemented with a finger clamp 4, during the use of which the patient's finger or toe is placed in the finger clamp 4 secured to the measuring probe 2 of the converter 10 using neodymium magnets. The finger clamp 4 maintains the patient's finger or toe so that it does not move and so that it remains in the same position in the course of the examination.

### Example 4

### System for the Examination of Nails with Transillumination

The system according to example 4 is provided with a transillumination attachment 5.

The electronics of the transillumination attachment 5 are connected to the microcontroller located in the housing 3 and the cable connected to the transillumination attachment 5 runs out next to the measuring probe 2.

The transillumination attachment 5 secured to the finger clamp 4 from below with neodymium magnets has one four-channel (red, green, blue, white) LED light source 23.

The lifting wedge 41 of the finger clamp 4 is placed under the patient's fingertip so that the slot 41a is located above the light source 23 of the transillumination attachment 5.

### Example 6

### System for the Examination of Nails with Transillumination

The system according to example 5, where the transillumination attachment 5 has a red LED light source 23.

### Example 7

### Combination Effect in the Case of Nail Fungus

The following table shows the ratio of the identification of nail fungus performed by the system according to example 1 in the case of the individual colour combinations (the value 1 corresponds to 100%, i.e. every case was identified).

Nail fungus vs. Healthy nails

| | normal | normal | normal | normal | normal | normal | polarised | polarised | polarised | polarised | polarised | polarised |
|---|---|---|---|---|---|---|---|---|---|---|---|---|
| Type | white | red | green | blue | UV | yellow | white | red | green | blue | uv | yellow |
| normal | 0.54 | 0.54 | 0.57 | 0.59 | 0.58 | 0.61 | 0.56 | 0.57 | 0.55 | 0.64 | 0.69 | 0.56 |
| white | | | | | | | | | | | | |
| normal | 0.54 | 0.61 | 0.67 | 0.6 | 0.61 | 0.69 | 0.61 | 0.58 | 0.64 | 0.67 | 0.56 | 0.64 |
| red | | | | | | | | | | | | |
| normal | 0.57 | 0.67 | 0.64 | 0.61 | 0.6 | 0.61 | 0.62 | 0.71 | 0.58 | 0.59 | 0.62 | 0.6 |
| green | | | | | | | | | | | | |
| normal | 0.59 | 0.6 | 0.61 | 0.61 | 0.62 | 0.58 | 0.57 | 0.64 | 0.61 | 0.58 | 0.6 | 0.53 |
| blue | | | | | | | | | | | | |
| normal | 0.58 | 0.61 | 0.6 | 0.62 | 0.64 | 0.56 | 0.58 | 0.61 | 0.65 | 0.53 | 0.58 | 0.61 |
| uv | | | | | | | | | | | | |
| normal | 0.61 | 0.69 | 0.61 | 0.58 | 0.56 | 0.6 | 0.64 | 0.62 | 0.65 | 0.61 | 0.55 | 0.62 |
| yellow | | | | | | | | | | | | |
| polarised | 0.56 | 0.61 | 0.62 | 0.57 | 0.58 | 0.64 | 0.61 | 0.62 | 0.74 | 0.69 | 0.55 | 0.69 |
| white | | | | | | | | | | | | |
| polarised | 0.57 | 0.58 | 0.71 | 0.64 | 0.61 | 0.62 | 0.62 | 0.67 | 0.6 | 0.54 | 0.64 | 0.58 |
| red | | | | | | | | | | | | |
| polarised | 0.55 | 0.64 | 0.58 | 0.61 | 0.65 | 0.65 | 0.74 | 0.6 | 0.54 | 0.64 | 0.61 | 0.61 |
| green | | | | | | | | | | | | |
| polarised | 0.64 | 0.67 | 0.59 | 0.58 | 0.53 | 0.61 | 0.69 | 0.64 | 0.64 | 0.69 | 0.57 | 0.64 |
| blue | | | | | | | | | | | | |
| polarised | 0.69 | 0.56 | 0.62 | 0.6 | 0.58 | 0.55 | 0.55 | 0.54 | 0.61 | 0.57 | 0.64 | 0.56 |
| UV | | | | | | | | | | | | |
| polarised | 0.56 | 0.64 | 0.6 | 0.53 | 0.61 | 0.62 | 0.69 | 0.58 | 0.61 | 0.64 | 0.56 | 0.58 |
| yellow | | | | | | | | | | | | |

The label of the first row is normal white image, in addition to which other images were made with the illumination types in the other columns, if then these were added to it and the neural network was trained in this, then we obtain the FScore contained in the cells (https://deepai.org/machine-learning-glossary-and-terms/f-score).

The first element means that if normal (not polarised) white light is combined with normal white light, then a not-too-good grade is obtained for this training task, a similarly not-so-good result is obtained when normal white light is combined with the normal red light found in the field next to it. However, the last but one cell of the first row shows that if images illuminated with normal white light are mixed with those illuminated with polarised UV light, then the separation of patients with nail fungus and healthy persons is at a comprehensible level, even in this limited data set.

In the case of the identification of other diseases such a table is of great assistance in the determination of the structure of the specific hardware, and can even be of assistance in the processing, as we see these results, then on the basis of these more serious training may be performed in a more targeted way. As each field in the table, including the main diagonal and the region above it, was an instance of neural network training.

### Example 8

### Results for the Recognition of Nail Diseases

The diseases of the nail were also labelled in order to support the diagnosis, in this way numerous photographs were available for diseases affecting nine different nails, as well as photographs of healthy nails, as a tenth class. During the training process the EfficientNet B2 model was used, and checking the effect of the complexity of the model an EfficientNet B3 model was also trained in the case of leukonychia and psoriasis. When constructing the training set on the basis of earlier experience it became apparent that if not only a single, multi-class model is trained but one model for each class, then we obtain a more successful training system. The FScore values displaying the training parameters and the success of the trained model are summarised in the following table:

| | | | | | | | Recognition | |
|---|---|---|---|---|---|---|---|---|
| | | Annotation - | | Train | | Test | normal | |
| | Annotation - | Everything | Train | the | Test | the | white | |
| Elements | Disease | else | Disease | rest | Disease | rest | (FScore) | Remark |
| Nail fungus | 31 | 2131 | 28 | 1918 | 3 | 213 | 0.87 | B2 |
| Psoriasis | 506 | 1656 | 455 | 1490 | 51 | 166 | 0.77 | B2 |
| Brittle Nail | 280 | 1882 | 252 | 1694 | 28 | 188 | 0.72 | B2 |
| Eczema | 109 | 2053 | 98 | 1848 | 11 | 205 | 0.84 | B2 |
| Nail Naevus | 43 | 2119 | 39 | 1907 | 4 | 212 | 0.58 | B2 |
| Leukonychia | 245 | 1917 | 221 | 1725 | 25 | 192 | 0.76 | B2 |
| Alopecia | 30 | 2132 | 27 | 1919 | 3 | 213 | 1 | B2 |
| Lichen | 59 | 2103 | 53 | 1893 | 6 | 210 | 0.7 | B2 |
| Planus | | | | | | | | |
| Raynaud | 24 | 2138 | 22 | 1924 | 2 | 214 | 0.66 | B2 |
| Disease | | | | | | | | |
| Normal nail | 835 | 1327 | 752 | 1194 | 84 | 133 | 0.74 | B2 |
| Leukonychia | 245 | 1917 | 221 | 1725 | 25 | 192 | 0.86 | B3 |
| Psoriasis | 506 | 1656 | 455 | 1490 | 51 | 166 | 0.83 | B3 |

The results are promising, however the number of elements of the training examples is still low, because the total number of elements of the database containing the nine different nail diseases plus the healthy class is 2162, in addition the occurrence of the diseases in no respect displays an even distribution either.

Among the results the binary grade of the normal nail may be used directly, as from the photograph of a nail this is able to determine whether one of the nine types of nail disease examined here can be seen or whether it depicts a healthy nail. Such a simplified grading process may even be used by a non-diagnostic service provider, such as a pedicurist, who by identifying the disease may make a recommendation for the client to consult a doctor.

### Example 9

### Results for the Identification of Non-Nail Diseases

In this experiment we attempted to identify the diagnosed underlying diseases of the patients only on the basis of photographs of their nails. The photographs to be analysed were taken using normal (not polarised) white illumination and a total of 2450 records were used. Exceptions from this were the first two diseases, which were trained on a narrowed down database. Binary classes were made here also, the purpose of which was only to identify or exclude individual underlying diseases. The model used for the training was the EfficientNet B2 and the training parameters and its success rate with the grading FScore value may be found in the following table:

| | | Annotation - | | Train | | Test | Recognition |
|---|---|---|---|---|---|---|---|
| | Annotation - | Everything | Train | the | Test | the | normal white |
| Elements | Disease | else | Disease | rest | disease | rest | (Eff B2, FScore) |
| Hypertension | 774 | 421 | 697 | 379 | 77 | 42 | 0.93 |
| Psoriasis | 391 | 804 | 352 | 724 | 39 | 80 | 0.90 |
| NIDMM | 319 | 2131 | 287 | 1918 | 32 | 213 | 0.77 |
| Heart failure | 378 | 2072 | 340 | 1865 | 38 | 207 | 0.82 |
| Kidney failure | 261 | 2189 | 235 | 1970 | 26 | 219 | 0.84 |
| Hepatomegaly | 155 | 2295 | 140 | 2066 | 16 | 230 | 0.74 |
| Eczema | 102 | 2348 | 92 | 2113 | 10 | 235 | 0.96 |
| Fatty liver | 116 | 2234 | 104 | 2011 | 12 | 223 | 0.73 |
| disease | | | | | | | |
| Liver fibrosis | 99 | 2351 | 89 | 2116 | 10 | 235 | 0.82 |
| Psoriatic arthritis | 100 | 2350 | 90 | 2115 | 10 | 235 | 0.84 |

### Example 10

### Method for the Optical Examination of Human Nails

Using the system according to example 5 an android telephone was inserted into the socket 31 of the converter 10. Following this, the patient's finger was secured under the measuring probe 2 using a finger clamp. The focus was adjusted so that the photographs would be sharp. Following this photographs are taken using the six-channel light sources 23 with the sequential use of the red, green, blue, white, yellow and UV lights in polarised illumination mode, non-polarised illumination mode, and large focus illumination mode. In other words at least six photographs are taken in the case of each illumination mode with the use of each of the individual channels. Furthermore, photographs are also taken in transillumination mode using each of the channels of the four-channel light source 23.

Following this, the photographs are sent through the cloud to the neural network, which was trained in advance using binary training. In other words, specialists attached labels onto the photographs in the database produced in advance or onto areas marked by specialists in the photographs about the formations visible there, such as bruising, nail fungus, etc. In addition the patients' known diseases were also attached to the photographs taken of the nail. During the binary training the neural network was informed whether the given label could be assigned to a photograph or to an area of it or not. Neural networks training in this way examine the photographs taken during the method and determine the state of health of the patients.

The advantage of the invention is that with the use of the neural network there is no need for the involvement of a specialist with great knowledge and experience in order to diagnose the various diseases from the condition of the nails.

A further advantage of the invention is that by using various illumination modes and light sources 23 that emit light of various wavelengths, various structural elements of the nail and the areas around the nail can be better depicted, and so in this way minute abnormalities in their early stages may also be easily detected.

A further advantage of the invention is that it may be used safely, as with the use of the finger clamp 4 there is only a minimal chance of the light emitted by the light sources 23 getting into the eyes of the patient or operating personnel.

1. System for the optical examination of human nails that has a camera and a power source or a smartphone provided with a connection to these, a converter (10) connected to the smartphone, which converter (10) contains a housing (3) and a measuring probe (2) connected to it, the housing (3) contains a microcontroller, the measuring probe (2) contains at least one macro lens (22) and at least one polarising filter (21), at least two light sources (23) and a polarising filter film (24) in front of some of the light sources (23) arranged in such a way that the axes of polarisation of the polarising filter films (24) and of the polarising filter (21) are rotated by 90 degrees as compared to each other, characterised by that as a result of the various light sources (23) the system is adapted to use various illumination modes and the smartphone is connected to a neural network.

2. System according to claim 1, characterised by that it contains a finger clamp (4) connected to the measuring probe (2) adapted for securing a finger or a toe.

3. System according to claim 2, characterised by that it contains a transillumination attachment (5) with an additional light source (23), which transillumination attachment (5) is connected to the microcontroller in the housing (3) and connected to the finger clamp (4) on the side opposite the nail of the finger to be clamped.

4. System according to claim 1, characterised by that the measuring probe (2) contains at least two macro lenses (22).

5. System according to claim 1, characterised by that the light sources (23) are multichannel light sources (23), preferably at least three-channel light sources (23), more preferably at least four-channel light sources (23), most preferably at least six-channel light sources (23).

6. System according to claim 5, characterised by that the colours of the multichannel light sources (23) are selected from among the following: red, green, blue, white, yellow, UV.

7. System according to claim 1, characterised by that at least six, preferably eight light sources (23) are located in the measuring probe (2).

8. System according to claim 1, characterised by that polarising filter film (24) is placed in front of a half of the light sources (23) located in the measuring probe (2), or in the case of an odd number of light sources (23) one more or one less of the light sources (23) has a polarising filter film (24) than the number of light sources (23) without a polarising filter film (24).

9. Method for the optical examination of fingernails using the system according to claim 1, characterised by that the method contains the following steps:
a) connecting the smartphone to the converter (10) or using the converter (10) integrated with the smartphone;
b) placing the patient's finger or toe in an immobile position in front of the system's measuring probe (2) in a position suitable for taking photographs;
c) adjusting the focus directed at the nail of the finger or toe of the patient to be examined on the smartphone;
d) illuminating the fingernail or toenail to be examined using at least two different illumination modes and the taking of photographs of the fingernail or toenail while the various illumination modes are being applied;
e) sending the photographs to the neural network;
f) the neural network determining the patient's health condition on the basis of the database it has at its disposal on the basis of the photographs that have been taken.

10. Method according to claim 9, characterised by that the system is used and in step b) the patient's finger or toe is secured in an immobile position using a finger clamp (4).

11. Method according to claim 9, characterised by that the system according is used and one of the at least two different illumination modes used in step d) is the transillumination mode.

12. Method according to claim 9, characterised by that the system is used and during step d) photographs are also taken in an illumination mode with large focus.

13. Method according to claim 9, characterised by that the system is used and in step d) a multichannel light source (23) is used in the case of the individual illumination modes.
